(19) United States
(12) Patent Application Publication
(10) Pub. No.: US 2023/0064767 A1
(43) Pub. Date: Mar. 2, 2023

(54) METHOD AND DEVICE FOR IDENTIFYING WORKPIECES

(71) Applicant: HOMAG GmbH, Schopfloch (DE)

(72) Inventor: Achim HOMEIER, Pfalzgrafenweiler (DE)

(21) Appl. No.: 17/799,223

(22) PCT Filed: Feb. 11, 2021
(86) PCT No.: PCT/EP2021/053313

§ 371 Date: Aug. 11, 2022

(30) Foreign Application Priority Data
Feb. 12, 2020 (DE) 10 2020 103 580.2

(51) Int. Cl. B23Q 15/12; G01N 33/46

(52) U.S. Cl. CPC B23Q 15/12; G01N 33/46

(57) ABSTRACT

The invention relates to a method for identifying workpieces, which preferably consist at least partly of wood, wood materials or the like, in a machining process, having the steps of: detecting at least one workpiece property prior to a machining step, uniquely assigning a workpiece category to the workpiece using the at least one detected workpiece property, and machining the workpiece in the machining step which is determined by the assigned workpiece category of the workpiece.

### TECHNICAL FIELD

The invention relates to a method and a device for identifying workpieces during a machining process, wherein the workpieces preferably consist of wood, wood materials or the like.

### PRIOR ART

In general, workpieces, which consist for example of wood, wood materials or the like, must be detected and labelled during a machining process. In the field of the furniture and structural component industry, in the method for labelling workpieces of the type mentioned at the outset, label carriers such as barcodes, transponders (RFID) and microchips are often used.

In this case, the labelling usually serves to uniquely identify a workpiece in the course of production. In addition, workpiece properties such as dimension, color and material can also be stored on the label carrier. Linking the information on the label carrier to corresponding routines in the machining process makes it possible for the machining of the workpieces to take place largely in an automated manner.

However, in the case of label carriers being applied to the surface of a workpiece, there is the risk that said carriers will be lost or damaged in the further machining process. Furthermore, the label carriers often have to be removed following the machining process since they may be disruptive for example from an aesthetic or productional perspective. Furthermore, the application and detection of the label carriers are in each case associated with additional measures in terms of apparatus.

For example, EP2230626 discloses a method for labelling solid planar workpieces by applying an RFID tag, in particular in a recess, to be made, of the workpiece, wherein the introduced RFID tab is provided with a coating for protection against the environment.

DE102014208746 discloses a further method for labelling workpieces, having the steps of applying a codable coating, in particular foil, edge and/or adhesive, to a surface of the workpiece, as well as reversible or irreversible coding of the coating by means of a coding unit, such that the coding can be read out again by a reader unit.

In the case of machining workpieces which preferably consist of wood, wood materials or the like, problems furthermore arise if the dimensions of the workpieces change, for example due to external influences such as temperatures, and subsequently the workpieces are positioned imprecisely in a machine tool and as a result the machining step is performed incorrectly. However, by means of electronic detection devices, workpiece properties can be detected upstream of the machine tool, and the machining step can be adjusted accordingly.

EP2308659 discloses a device for machining workpieces, which a conveying device for conveying the workpieces, and an electronic detection device for detecting a position of an optical and/or three-dimensional structure of the workpieces conveyed on the conveying device.

In the case of incorrect performance of a machining step, the following machining step may be influenced in a disadvantageous manner, or the incorrectly machined workpiece may leave the machining process unnoticed.

### DESCRIPTION OF THE INVENTION

The object of the invention is that of providing a method, and a device provided therefor, for identifying workpieces, which allow for control and a quality check of the workpieces in the machining process, wherein the required outlay with respect to apparatus should be kept low.

This object is achieved according to the invention by a method for identifying workpieces having the features of claim 1 and by a device for carrying out the method according to claim 10. Advantageous embodiments and improvements of the invention can be found in the dependent claims.

The invention is based in particular on the finding that, in the case of identification and quality checking of workpieces in a machining process, synergistic effects can be used, which reduces the outlay, originally required for this, with respect to apparatus. It has furthermore been found that, in order to control and monitor a machining process comprising different workpieces, it is not necessarily the case that each workpiece has to b e labelled individually. For this purpose, in the case of the present method, the workpieces are identified on the basis of their workpiece properties. If the workpiece properties for the workpiece identification correspond to the workpiece properties for the workpiece check, then both processes can be carried out using the same detection system. In this connection, the detection of geometric and/or physical workpiece properties has been found to be particularly suitable.

Using these findings, the invention provides a method for identifying workpieces, which preferably consist at least partly of wood, wood materials, or the like, in a machining process, in which at least one workpiece property is detected before a machining step, in order to uniquely assign the workpiece to a particular workpiece category by means of said property. The workpiece is subsequently machined in the machining step in accordance with the assigned workpiece category.

The invention thus ensures that identification of the workpiece can take place exclusively on the basis of the workpiece properties themselves. The identification data of the workpiece are consequently inseparably linked to the workpiece. As a result, the workpiece can be identified in the machining process at any time. Additional application of label carriers is accordingly not required.

Furthermore, this results in a dynamic machining process since a machining step is adjusted in a situational manner, according to the existing workpiece properties of the workpiece to be machined, and is not restricted to defined data of a label carrier.

Furthermore, the advantage results that the at least one detected workpiece property, which is used for assignment to a corresponding workpiece category, is also simultaneously the property of a workpiece which is intended to be checked for possible deviations from a standard. The synergistic effect consequently consists in the detected workpiece property for workpiece identification and the workpiece property to be checked corresponding. The outlay with respect to apparatus, during the detection of workpiece properties for workpiece identification and a workpiece check can be restricted to the same detection system.

The method according to the invention is preferably carried out such that at least one workpiece property to be changed is detected after the machining step, and a subsequent process step is determined on this basis. This ensures that the workpiece is correctly machined in accordance with the machining step. In this case it is advantageous for the next process step to be dependent on the machining result of the previous machining step.

It is furthermore advantageous for information relating to the machining success of the workpiece to be generated and associated therewith. The machining process can be controlled more effectively using the additionally obtained information relating to the machining success of the workpieces.

The method according to the invention according to which, in the subsequent process step, the workpiece is further machined or discharged from the machining process, i s particularly preferred. This is advantageous in that only correctly machined workpieces remain in the machining process, and subsequent machining steps are not influenced by incorrectly machined workpieces. Workpieces having irreversible machining errors do not remain unidentified in the machining process and are discharged. In contrast, workpieces having reversible machining errors can be fed to additional machining in a further machining step.

In a further advantageous embodiment of the method of the invention, prior to the machining step, additionally the at least one workpiece property to be changed is detected and the workpiece is machined according to the allocated workpiece category and the additionally detected workpiece property to be changed.

The detection of at least one workpiece property to be changed prior to the machining step makes it possible for a workpiece to also pass through a machining step multiple times. This is advantageous in that, in the case of repeated passage through a machining step, in each case different workpiece properties can be changed, and it is not the case that the same machining step is carried repeatedly.

The present method of the invention is in particular characterized by the workpiece being assigned a workpiece category by combining a plurality of the same and/or different workpiece properties.

Consequently, depending on the detected number of the same and/or different workpiece properties a corresponding number of definable workpiece categories are available, into which the workpieces can be assigned. In this case, it is advantageous that increasing the number of the detected workpiece properties makes it possible for the number of assignable workpiece categories to be increased accordingly.

In a further preferred embodiment of the method, physical and/or geometric workpiece properties are detected. These can for example be weight specifications, dimensions, colors or three-dimensional structures of the workpieces.

The physical and/or geometric workpiece properties offer a plurality of different value sets that are to be detected. This makes it possible to provide a data pool having a plurality of workpiece properties, and to combine therefrom a plurality of different workpiece categories, on the basis of which the workpieces are identified.

In a further preferred embodiment, the workpiece properties to be detected and the workpiece categories are defined in a data pool.

As a result, a data pool of workpiece properties and workpiece categories is defined from a plurality of workpiece properties and workpiece categories. It is advantageous that flexibly different data pools of workpiece properties and workpiece categories can be specified for different machining processes having different workpieces, on the basis of which it is possible to identify different workpieces.

The present method is particularly preferred in which the workpiece properties detected during the machining process are additionally stored in the data pool.

In this way, a data pool of workpiece properties and generated, which corresponds to the workpieces contained in the machining process. This is advantageous in that real-time data relating to the workpieces in the machining process, and the properties thereof, are available.

It is furthermore preferable for a process control system to access the data pool and control the machining process.

This makes it possible for individual machining steps of the machining process to be adjusted to one another, and for the machining process to be optimally utilized. Furthermore, the process control system allows the user an overview of the workpieces already in the machining process, and allows for a flexible design of a superordinate production process.

The present invention furthermore provides a device for carrying out the method, comprising at least one detection system which is configured upstream of a workpiece machining unit, in order to detect workpiece properties which are necessary for assigning a workpiece category, and at least one workpiece machining unit which is configured to machine the workpiece in accordance with an assigned workpiece category.

The invention thus ensures that the machining step to be performed is specified upstream of the machining unit, on the basis of the assigned workpiece category. This is advantageous in that a label carrier can be omitted. In addition to the detection of the workpiece properties for identifying the workpieces, the detection system additionally allows for said workpiece properties to be checked. This is advantageous in that the outlay with respect to apparatus is restricted to one detection system.

A machining process having individual machining steps can furthermore take place dynamically since a machining step is adjusted in a situational manner according to the detected workpiece properties, and is not dependent on the data of a label carrier.

In a preferred embodiment of the device, at least one detection system is configured downstream of the workpiece machining unit, in order to detect the workpiece property that is to be changed.

This ensures that the workpiece is correctly machined in accordance with the machining step, and additional information relating to the machining success of the workpiece is generated.

In an advantageous embodiment of the device, at least one detection system is configured upstream of the workpiece machining unit, in order to detect the workpiece property that is to be changed.

In this way, a detection is made as to whether machining has to be carried out, according to the allocated workpiece category, in the machining unit. This is advantageous in that one machining unit can perform different machining steps on a workpiece.

Furthermore, in a preferred embodiment of the device, the detection system comprises one and/or more active and/or passive sensors, in particular mechanical, thermoelectric, resistive, piezoelectric, capacitive, inductive, optical, acoustic or magnetic sensors. A plurality of geometric and/or physical workpiece properties can thus be detected. In this case, it is advantageous that the sensors can be selected individually according to the properties of the workpieces to be machined.

In a particularly preferred embodiment of the device, a switch element is configured downstream of the machining unit, in order to discharge the workpiece or to feed it to a further machining step.

The switch element makes it possible for incorrectly machined workpieces to either be discharged from the machining process or to be fed again to a machining step. This is advantageous in that said workpieces do not have to be immediately removed by hand or relocated to another location in the machining process. This prevents incorrectly machined workpieces being a drain on the capacities of further machining steps.

More preferably, the device comprises a process control system in order to control the machining process on the basis of the stored data pool of the detected workpiece properties and the assigned workpiece categories.

This makes it possible for individual machining steps of the machining process to be adjusted to one another, and for the machining process to be optimally utilized as a result. Furthermore, the process control system allows the user an overview of the workpieces already in the machining process, and as a result allows for a flexible design of a superordinate production process.

### BRIEF DESCRIPTION OF THE DRAWINGS

Further features and advantages of a device, a use and/or a method can be found in the following description of embodiments, with reference to the accompanying drawings. In said drawings:

FIG. 1: is a schematic view of a device for identifying workpieces in a machining process;

FIG. 2: is a schematic view of a device for identifying a door and a tabletop in a machining process which includes, by way of example, the machining steps of varnishing and edge banding.

### DESCRIPTION OF THE EMBODIMENTS

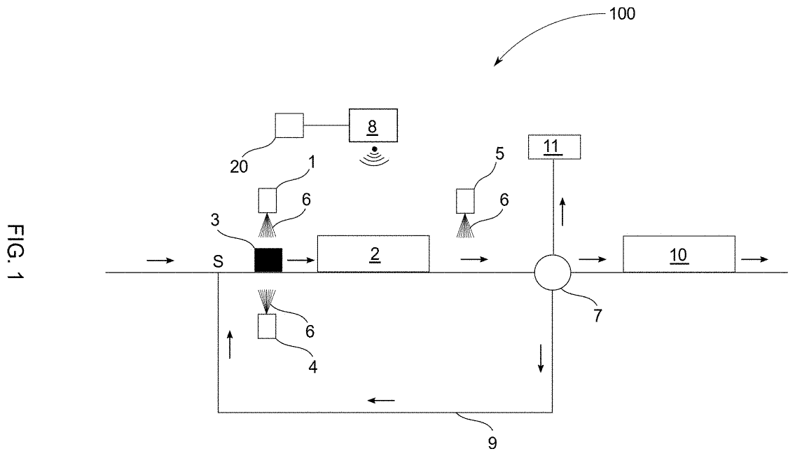

FIG. 1 is a schematic view of a device 100 for identifying workpieces 3 in a machining process according to a first embodiment of the invention.

The workpieces consist for example of wood, wood materials, synthetic materials, metal or the like. The workpieces 3 are in particular known from furniture manufacture, interior furnishings, in vehicle construction and shipbuilding, in house construction and interior fittings and the like.

The device 100 comprises one or more machining units for machining the workpieces. For example the machine tools and coating machines, such as form at and edge banding machines, varnishing machines, broadband grinders, planning machines, drills and dowel insertion machines, plate-splitting saws and the like are provided as machining units.

In particular, the device shown is such that two detection systems 1, 4 are configured upstream, and a detection system 5 is configured downstream, of the machining unit 2.

The detection systems 1, 4, 5 are equipped with various sensors such as mechanical, thermoelectric, resistive, piezoelectric, capacitive, inductive, optical, acoustic or magnetic sensors.

In the present embodiment, a conveying device 9 is configured such that it allows for a closed conveying circuit of a workpiece 3.

The conveying device 9 for conveying the workpieces 3 is configured for example in the form of band conveyors, conveyor belts, conveyor chains, driverless transport systems (DTS), autonomous handling systems, robots and the like.

The workpieces 3 are fed to the conveying circuit again at a location S and it is possible to regulate, by means of a switch element 7, whether these remain in the conveying circuit, are discharged into an external storage unit 11, or are transported to a further machine tool 10. The switch element 7 is preferably arranged directly downstream of the third detection system 5.

Furthermore, the device 100 is configured such that the location S, at which the workpieces 3 are transported, via the switch element 7, back to the machining unit 2 and at which the unmachined workpieces 3 are discharged into the machining process, is located upstream of the first 1 and second detection system 1, 4.

The device 100 is in particular configured such that a central process control system 8 controls the individual units (1, 2, 4, 5, 6, 7, 9, 11) of the device 100, for example by means of a wireless connection. The device 100 described in FIG. 1 can be considered a distinct process unit. In order to control the machining process, the process control sy stem 8 can access a data pool 20.

According to further embodiments which are not shown, an entire process consists of process units connected in series and/or in parallel, as a result of which a more continuous and gap-free machining process of a workpiece 3 is made possible.

In the following, the method will now be described by way of example with reference to the device 100 shown in FIG. 1.

In a first step of the claimed method, first of all the workpiece properties, specified for identification, of the workpiece 3 which is loaded into the machining process at the location S and transported, via the conveying device 9, to the first detection system 1, are detected by means of a sensor 6 of the detection system 1.

The workpiece properties suitable for identification are in particular geometric and/or physical properties. Depending on the geometric and physical properties of the workpiece 3 to be machined, a plurality of different workpiece properties results, which properties can be detected in the present machining process. The workpiece properties to be detected are defined in a data pool 20.

In the second method step, the workpiece 3 is uniquely assigned a workpiece category, on the basis of the detected workpiece properties, and identified thereby. A workpiece category is defined in the data pool 20 by a workpiece property or by a plurality of workpiece properties, according to the principle of combinatorics. For example, a workpiece category can be defined by a surface pattern in combination with a weight specification. In particular, in this case, it is also possible to define a value set for the weight specification, instead of a single value for the weight specification.

The assignment of the workpiece 3 to a workpiece category is performed in particular by the process control system 8, in that it accesses the data pool 20. In this case, the process control system 8 compares the values of the workpiece properties of the workpiece 3, detected by means of the detection system 1, with the defined values of the workpiece properties of the defined workpiece categories. If the detected values of the workpiece properties of the workpiece 3 correspond to the defined values of the workpiece properties of a workpiece category, then the process control system 8 assigns the workpiece 3 to this workpiece category, as a result of which it is identified.

The process control system 8 furthermore checks the detected values of the workpiece properties of the workpiece 3, for example also for possible deviations from a defined standard. FIG. 1 does not show that a further switch element may be configured upstream of the machining unit 2, which discharges the workpiece 3 from the machining process if it does not correspond to a standard.

After the unambiguous assignment of the workpiece 3 to a workpiece category, and a positive check of the workpiece properties for adherence to a standard, the third method step is initiated. In this step, the workpiece 3 is machined, in the machining unit 2, according to the assigned workpiece category. In the process control system 8, in particular for each workpiece category one or more operations are stored by the machining unit 2. The process control system 8 determines the machining, to be performed, of the workpiece 3 in the machining unit in a situational manner, according to the workpiece category.

After machining of the workpiece 3 in the machining unit 2, a third detection system 5 detects the changed workpiece properties of the workpiece 3 by means of a sensor 6. Said properties are transmitted to the process control system 8 and stored in the data pool 20. Depending on the machining result of the machining unit 2, the process control sy stem 8 controls the conveyance of the workpiece 3 by means of the conveying unit 9 via the switch element 7, either back to the first machining unit 2, or into a storage unit 11 or to a further machining unit 10.

Repeated feeding of the workpiece 3 to the first machining unit 2 takes place, for example, if the workpiece 3 was not machined competently, the error is reversible, and the machining can be performed again. A further machining step can also be performed using the same machining unit 2, at another and/or the same location on the workpiece 3, such that a further workpiece property is changed. In this case, in particular the machining unit 2 is configured such that the workpiece 3 can be re-aligned in the machine tool 2.

If the machining result is incorrect and the error is irreversible or the machining has been terminated, the workpiece 3 is stored in the storage unit 11 of the device.

If the workpiece 3 is fed repeatedly to the machining unit 2, in particular at least one second detection system may be provided upstream of the machining unit 2, in order to detect the changed workpiece properties of the workpiece 3. The work step in the machining unit 2 is determined and performed by the process control system on the basis of the assigned workpiece category and/or changed workpiece properties of the workpiece 3.

All data relating to the detected workpiece properties of the workpieces 3 during the machining process are stored in the data pool 20. Access of the process control system 8 to the data pool 20 allows the user to ascertain, by means of visualization, the location, in the machining process, at which a workpiece 3 of a particular category is located, and what the machining state is. If the machining process contains different workpieces 3, the process control system 8 can regulate the individual units 1, 2, 4, 5, 6, 7, 9, 10, 11 and dynamically control the entire process, on the basis of the data of the data pool 8.

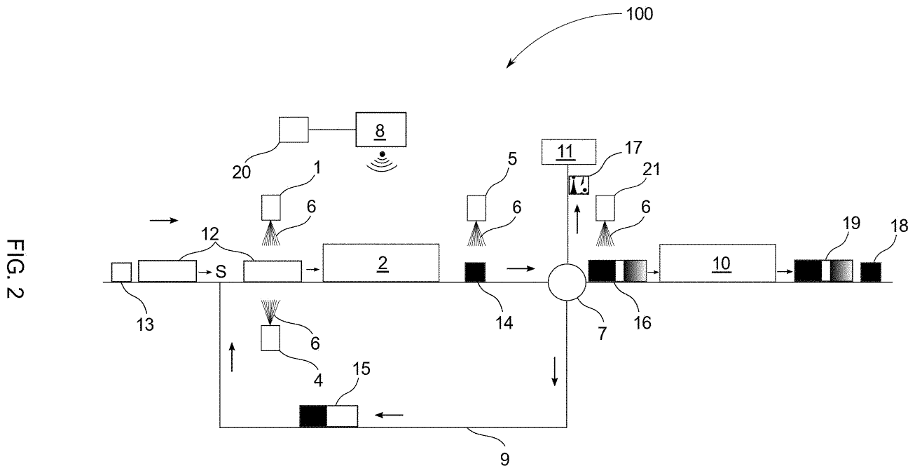

FIG. 2 is a schematic view of a device 100 for identifying a door 12 and a tabletop 13 in a machining process according to a second embodiment of the invention, which includes, by way of example, the machining steps of varnishing and edge banding. The claimed method will be described on the basis of a possible machining process from practice, with reference to FIG. 2.

At the location S of the device 100, two rectangular workpieces to b e machined, in the example set out here a semi-finished door 12 of a cupboard and a semi-finished tabletop 13, are loaded into the machining process. By means of a camera of a first optical detection system 1, the length and the height of the door 12 (X_{door}, Y_{door}) and of the tabletop 13 (X_{table}, Y_{table}) are detected. It is stored in the data pool 20 that, for a combination of a specified value range [x₁, . . . , xₙ] with respect to the length with a specified value range [y₁, . . . , yₙ] with respect to the height of a workpiece, either the workpiece category “door” or “table” is defined. The process control system 8 accesses the data pool 20 and assigns the workpiece “door”, for which a length X_{door} and height Y_{door} have been detected by the optical detection system 1, the category “door”, if the detected values in each case correspond to a value in the corresponding value range which is defined for the length and the height of the category “door”. The described process takes place analogously for the tabletop 13. The categories “door” and “table” are then uniquely distinguishable by means of the two value ranges for length and height, if at least one of the two value regions for length and height does not intersect. In the method described here, the value ranges are selected so as to be so narrow that they simultaneously also fulfil the function of checking the tabletop (12) and the door for deviations from a standard. The value range which is defined for the assignment of a workpiece to a category then corresponds to the allowable value range for a standard dimension.

After the unmachined tabletop and the door have been uniquely assigned the category “door” and “table”, the machining step stored in the process control system 8 for the respective workpiece category is performed, accordingly, in the varnishing machine 2.

The second optical detection system 5 identifies, by means of a camera 6, the varnishing region and the pattern of the varnishing of the door and the tabletop, and stores the information in the data pool 20. On the basis of this information, by means of controlling the switch 7, the process control system 8 causes the partially varnished door 15 to be transported back into the varnishing machine 2, a tabletop 17 to be discharged into a store 11 on account of incorrect varnishing, and a correctly varnished door 16 to be transported to the edge banding machine 10.

The second optical detection system 4, upstream of the varnishing machine 2, detects the partially varnished part of the door 15, wherein the process control system 8 assigns the door (15) the category “door”, by means of the first optical detection system 1 which detects the height and length of the door. For the category “door”, the process control system 8 initiates, on account of the additional information relating to the varnishing state of the door 15, a further varnishing process on the door by the varnishing machine 2.

The method for identifying the door 16 and the tabletop 14 upstream of the edge banding machine 10 takes place in a manner analogous to the method described above. A door 19 and the tabletop 18 is provided with an edge, by means of the edge banding machine 10, in accordance with the workpiece category.

### LIST OF REFERENCE NUMBERS

1 detection system, optical detection system

2 machining device, varnishing machine

3 workpieces

4 detection system, optical detection system

5 detection system, optical detection system

6 sensor, camera

7 switch element

8 process control system

9 conveying device

10 machining device, edge banding machine

11 storage unit

12 door

13 tabletop

14 varnished tabletop

15 partially varnished door

16 varnished door

17 incorrectly varnished tabletop

18 varnished tabletop having an edge

19 varnished door having an edge

20 data pool

21 optical detection system

1. A method for identifying workpieces, comprising wood, wood materials or the like, in a machining process, comprising the steps of:
detecting at least one workpiece property prior to a machining step,
uniquely assigning the workpiece to a workpiece category on the basis of the at least one workpiece property, and
machining the workpiece based on the workpiece category.

2. The method of claim 1, wherein at least one workpiece property to be changed is detected after the machining step, and a subsequent process step is determined on this basis.

3. The method of claim 2, wherein, in the subsequent process step, the workpiece is further machined or discharged from the machining process.

4. The method of claim 1, wherein, prior to the machining, at least one workpiece property to be changed is detected and the workpiece is machined based on the workpiece category and the at least one workpiece property to be changed.

5. The method of claim 1, wherein the workpiece is assigned to a workpiece category by combining a plurality of workpiece properties.

6. The method of claim 1, wherein physical or geometric workpiece properties are detected.

7. The method of claim 1, wherein the at least one workpeice property is one of a plurality of workpiece properties to be detected and the workpiece category is one of a plurality of workpiece categories defined in a data pool.

8. The method of claim 7, further comprising storing in the data pool the at least one workpiece property detected during the machining process.

9. The method of claim 8, wherein a process control system accesses the data pool and controls the machining process.

10. A device for identifying workpieces, comprising wood, wood materials or the like, the device comprising:
at least one detection system configured upstream of a workpiece machining unit, in order to detect workpiece properties which are necessary for assigning a workpiece category, and
at least one workpiece machining unit configured to machine the workpiece in accordance with an assigned workpiece category.

11. The device of claim 10, wherein the at least one detection system is configured upstream of the workpiece machining unit in order to detect workpiece properties to be changed.

12. The device of claim 11, further comprising at least one detection system downstream of the machining unit in order to detect changed workpiece properties.

13. The device of claim 10, wherein the detection system comprises one or more active or passive sensors.

14. The device of claim 11, further comprising a switch element configured downstream of the at least one detection system and the machining unit in order to discharge the workpiece or to feed it to a further machining step.

15. The device of claim 10, furhter comprising a process control system configured to control the machining process on the basis of the data pool.

16. The device of claim 13, wherein the one or more active or passive sensors includes mechanical, thermoelectric, resistive, piezoelectric, capacitive, inductive, optical, acoustic, or magnetic sensors.

17. The method of claim 6, wherein the physical or geometric workpiece properties include weight specifications, dimensions, colors, or three-dimensional structures.
